(19) United States
(12) Patent Application Publication
(10) Pub. No.: US 2006/0185098 A1
(43) Pub. Date: Aug. 24, 2006

(54) Composition for bleaching and simultaneously dyeing keratin fibers, comprising 7-(6'-methylphenylazo)-1-acetamido-3,6-disulfo-8-hydroxynaphthalene

(76) Inventors: Sylvain Kravtchenko, Asnieres (FR); Valerie Bonnardel, Colombes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/293,361

(22) Filed: Dec. 5, 2005

Related U.S. Application Data
(60) Provisional application No. 60/670,268, filed on Apr. 12, 2005.

(30) Foreign Application Priority Data
Dec. 3, 2004 (FR) 04 52862

(51) Int. Cl. A61K 8/00

(52) U.S. Cl. 8/405

(57) ABSTRACT

Disclosed herein is a composition for bleaching and simultaneously dyeing keratin fibers, comprising at least one dye chosen from 7-(6′-methylphenylazo)-1-acetamido-3,6-disulfo-8-hydroxynaphthalene and addition salts thereof, at least one peroxygenated salt, and at least one alkaline agent. Also disclosed herein is a method for bleaching and dyeing keratin fibers comprising applying this composition to the keratin fibers. The composition in accordance with the present disclosure is suitable for dark hair, exhibits improved stability over time, and allows chromatic and fast dyeing to be obtained.

This application claims benefit of U.S. Provisional Application No. 60/670,268, filed Apr. 12, 2005, the contents of which are incorporated herein by reference. This application also claims benefit of priority under 35 U.S.C. §119 to French Patent Application No. 04 52862, filed Dec. 3, 2004, the contents of which are also incorporated herein by reference.

Disclosed herein is a composition for bleaching and simultaneously dyeing keratin fibers, for example, human keratin fibers such as the hair, comprising at least one dye chosen from 7-(6′-methylphenylazo)-1-acetamido-3,6-disulfo-8-hydroxynaphthalene and addition salts thereof, at least one peroxygenated salt, and at least one alkaline agent.

When a person wishes to radically change the color of his or her hair, especially when he or she wishes to obtain a color lighter than his or her original color, it is often necessary to bleach and, where necessary, to dye the hair. Several methods exist for doing this.

The first method comprises using lightening products based on aqueous ammonia and hydrogen peroxide. These products may optionally contain dyes, which allow the hair to be lightened and simultaneously dyed. However, the lightening performance of these products remains limited, more particularly for applications to natural and/or dyed dark foundation colors.

The second method comprises applying to the hair a lightening composition based on peroxygenated salts such as persulfate and alkaline agents to which has been added hydrogen peroxide at the time of use, in order to obtain greater lightening. This type of product is very satisfactory and more suited to dark foundation color, but leads to only a very restricted range of tints. It is thus necessary to correct the shade obtained by applying, in a second stage, a dye product to the hair. This two-step process has the drawback of being relatively long.

To overcome this drawback, it is known practice to add dyes to these lightening products. This method allows the hair fiber to be dyed and simultaneously bleached. Since the level of lightening is substantial, it is particularly suited to natural and/or dyed foundation colors. However, there is a very limited number of dyes that are stable under these highly oxidative conditions, which limits the variety of tints that may be obtained. Moreover, this instability is reflected by a more or less rapid change in the tint during application, which leads to poorly reproducible results.

Furthermore, the fastness of these dyes with respect to external agents, for example, light and shampoo, is not always satisfactory.

Direct dyes of anthraquinone, azo, triarylmethane, thiazine, quinone, and nitro type, which are stable in these highly oxidative media, have been proposed in U.S. Pat. No. 5,688,291, International Publication No. WO 02/074 270, and German Patent No. 203 03 559. However, these dyes may be unsatisfactory in terms of chromaticity, fastness, and stability during the application time.

Thus it would be desirable to provide novel compositions for bleaching and simultaneously dyeing keratin fibers, for example, human keratin fibers such as the hair, which are suitable for dark foundation colors, which show good stability over time and/or which allow chromatic and fast colorations to be obtained.

Disclosed herein, therefore, is a composition for bleaching and simultaneously dyeing keratin fibers, comprising:
at least one dye chosen from 7-(6′-methylphenylazo)-1-acetamido-3,6-disulfo-8-hydroxynaphthalene and the addition salts thereof;
at least one peroxygenated salt; and
at least one alkaline agent.

The composition in accordance with the present disclosure may be suitable for bleaching and simultaneously dyeing dark hair. It may show improved stability over time and/or allow a chromatic coloration to be obtained. Furthermore, with suitable concentrations of dyes according to the present disclosure, pastel tints may be obtained.

This coloration is resistant to one or more of the various attacking factors to which hair may be subjected, such as shampoo, rubbing, light, bad weather, sweat, and permanent reshaping operations. It is also powerful, aesthetic, and/or, furthermore, sparingly selective, i.e., it produces only small differences between different parts of a hair or of a head of hair that are differently sensitized.

Also disclosed herein is a method for bleaching and simultaneously dyeing keratin fibers, comprising applying the composition described herein to the keratin fibers. Further disclosed herein are multi-compartment devices for implementing this method.

Dyes

7-(6′-Methylphenylazo)-1-acetamido-3,6-disulfo-8-hydroxynaphthalene and addition salts thereof are direct azo dyes. Non-limiting examples of the addition salts of 7-(6′-methylphenylazo)-1-acetamido-3,6-disulfo-8-hydroxynaphthalene that may be used in accordance with the present disclosure include the addition salts with an organic or mineral base, for example, the salts of alkali metals, the salts of alkaline-earth metals, and the salts of organic amines such as alkanolamines.

In at least one embodiment of the present disclosure, the at least one dye may be chosen from the sodium salts of 7-(6′-methylphenylazo)-1-acetamido-3,6-disulfo-8-hydroxynaphthalene, and mixtures thereof. In another embodiment, the at least one dye is chosen from Acid Red 35, also known as Supramine Red 3BA, the structure of which is as follows: 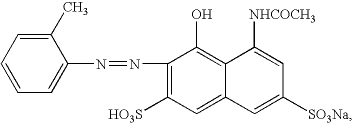
and Acid Red 55, the structure of which is as follows: 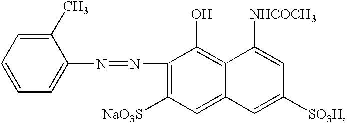
and the addition salts thereof.

7-(6′-methylphenylazo)-1-acetamido-3,6-disulfo-8-hydroxynaphthalene and/or addition salts thereof may be present in the composition in an amount ranging from 0.0001% to 10% by weight, for example, from 0.001% to 8%, or from 0.01% to 5% by weight relative to the total weight of the composition.

Peroxygenated Salts

The at least one peroxygenated salt that is useful in the present disclosure may be chosen, for example, from alkali metal persulfates, alkali metal perborates, alkali metal percarbonates, alkali metal peroxides, alkaline-earth metal persulfates, alkaline-earth metal perborates, alkaline-earth metal percarbonates, alkaline-earth metal peroxides, and mixtures thereof. In at least one embodiment, the at least one peroxygenated salt may be chosen from persulfates and mixtures thereof, for example, sodium persulfate, potassium persulfate, ammonium persulfate, and mixtures thereof.

The at least one peroxygenated salt may be present in the composition in an amount ranging from 10% to 70% by weight, for example, from 20% to 60% by weight relative to the total weight of the composition.

Alkaline Agents

The at least one alkaline agent that is useful in the composition of the present disclosure may be chosen, for example, from urea, ammonium salts, such as ammonium chloride, ammonium sulfate, ammonium phosphate, and ammonium nitrate, silicates, phosphates, and carbonates of alkali metals or of alkaline-earth metals such as lithium, sodium, potassium, magnesium, calcium, and barium, and mixtures thereof. In at least one embodiment, the at least one alkaline agent may be chosen from ammonium chloride, silicates, carbonates, and mixtures thereof.

The at least one alkaline agent may be present in the composition in an amount ranging from 0.01% to 40% by weight, for example, from 0.1% to 30% by weight relative to the total weight of the composition.

The composition in accordance with the present disclosure may be in the form of a powder or a paste. In one embodiment, the composition of the present disclosure may be in the form of a paste.

When the composition in accordance with the present disclosure is in the form of a paste, it may also comprise at least one inert organic liquid phase.

Inert Organic Liquid Phase

As used herein, the term “liquid phase” means any phase capable of flowing at room temperature, generally ranging from 15° C. to 40° C., and at atmospheric pressure, under the action of its own weight.

Examples of inert liquid phases include, but are not limited to, the polydecenes of formula C₁₀ₙH_{[(20n)+2]} in which n ranges from 3 to 9, for example, from 3 to 7, esters of fatty alcohols, esters of fatty acids, C₁₂-C₂₄ fatty acid esters of sugars, C₁₂-C₂₄ fatty acid diesters of sugars, cyclic ethers, cyclic esters, silicone oils, mineral oils, plant oils, and mixtures thereof.

The compounds of formula C₁₀ₙH_{[(20n)+2]} with n ranging from 3 to 9 correspond to the name “polydecene” in the CTFA dictionary 7th edition, 1997 of the Cosmetic, Toiletry and Fragrance Association, USA, and also to the same INCI name in USA and Europe. These are products of hydrogenation of poly-1-decenes.

In at least one embodiment of the present disclosure, the inert liquid phase is a polydecene for which, in the formula, n ranges from 3 to 7.

Examples of suitable polydecenes include, but are not limited to, the product sold under the name Silkflo® 366 NF Polydecene by the company Amoco Chemical, and those sold under the names Nexbase® 2002 FG, 2004 FG, 2006 FG, and 2008 FG by the company Fortum.

Suitable esters of fatty alcohols and esters of fatty acids, include, but are not limited to:
esters of linear or branched, saturated lower C₃-C₆ monoalcohols with C₁₂-C₂₄ monofunctional fatty acids, these fatty acids possibly being linear or branched, and saturated or unsaturated, for example, those chosen from oleates, laurates, palmitates, myristates, behenates, cocoates, stearates, linoleates, linolenates, caprates, arachidonates, and mixtures thereof, for example, oleopalmitates, oleostearates, and palmitostearates. In at least one embodiment, the esters may be chosen from isopropyl palmitate, isopropyl myristate, and octyidodecyl stearate;
esters of linear or branched C₃-C₈ monoalcohols with C₈-C₂₄ difunctional fatty acids, these fatty acids possibly being linear or branched, and saturated or unsaturated, for instance the isopropyl diester of sebacic acid, also known as diisopropyl sebacate;
esters of linear or branched C₃-C₈ monoalcohols with C₂-C₈ difunctional fatty acids, these fatty acids possibly being linear or branched, and saturated or unsaturated, for instance dioctyl adipate and dicaprylyl maleate; and
the ester of a trifunctional acid, for instance triethyl citrate.

As regards the C₁₂-C₂₄ fatty acid esters and diesters of sugars, the term “sugar” means compounds comprising several alcohol functions, with or without an aldehyde or ketone function, and which comprise at least four carbon atoms. These sugars may be chosen from monosaccharides, oligosaccharides, and polysaccharides.

Non-limiting examples of sugars include sucrose (or saccharose), glucose, galactose, ribose, fucose, maltose, fructose, mannose, arabinose, xylose, and lactose, and derivatives thereof, for example, alkyl derivatives, such as methyl derivatives, for instance methylglucose.

Suitable fatty acid esters of sugars that may be used according to the present disclosure may be chosen from esters and mixtures of esters of sugars described above and of linear or branched, saturated or unsaturated C₁₂-C₂₄ fatty acids.

The esters may be chosen from mono-, di-, tri-, and tetraesters, polyesters, and mixtures thereof.

These esters may be chosen, for example, from oleates, laurates, palmitates, myristates, behenates, cocoates, stearates, linoleates, linolenates, caprates, and arachidonates, and mixtures thereof, for instance mixed oleo-palmitates, oleo-stearates, and palmito-stearates.

In at least one embodiment of the present disclosure, the esters may be chosen from mono- and diesters, for example, sucrose, glucose, and methylglucose, mono- and dioleates, stearates, behenates, oleopalmitates, linoleates, linolenates, and oleostearates.

A non-limiting example of a suitable commercial product is the product sold under the name Glucate® DO by the company Amerchol, which is a methylglucose dioleate.

Other suitable examples of esters and mixtures of esters of sugar and of fatty acid include, but are not limited to:

the products sold under the names F160, F140, F110, F90, F70, and SL40 by the company Crodesta, respectively denoting sucrose palmito-stearates formed from 73% monoester and 27% diester and triester; 61% monoester and 39% diester, triester, and tetraester; 52% monoester and 48% diester, triester, and tetraester; 45% monoester and 55% diester, triester, and tetraester; and 39% monoester and 61% diester, triester, and tetraester; and sucrose monolaurate;

the products sold under the name Ryoto Sugar Esters, for example referenced B370 and corresponding to sucrose behenate formed from 20% monoester and 80% di-triester-polyester;

sucrose mono-di-palmito-stearate sold by the company Goldschmidt under the name Tegosoft® PSE.

Non-limiting examples of cyclic ethers and cyclic esters include γ-butyrolactone, dimethyl isosorbide, and diisopropyl isosorbide.

Silicone oils may also be used as an inert organic liquid phase.

In at least one embodiment, suitable silicone oils may be chosen from liquid, non-volatile silicone fluids with a viscosity of less than or equal to 10 000 mPa·s at 25° C., the viscosity of the silicones being measured according to ASTM standard 445 Appendix C.

Silicone oils are defined, for example, in greater detail in Walter Noll's “Chemistry and Technology of Silicones” (1968)—Academic Press.

Examples of silicone oils that may be used according to the present disclosure include, but are not limited to, the silicone oils sold under the names DC-200 Fluid—5 mPa·s, DC-200 Fluid—20 mPa·s, DC-200 Fluid—350 mPa·s, DC-200 Fluid—1000 mPa·s, and DC-200 Fluid—10 000 mPa·s by the company Dow Corning.

Mineral oils may also be used as an inert organic liquid phase, for instance liquid paraffin.

Plant oils may also be suitable for use, for example, avocado oil, olive oil, and liquid jojoba wax.

In at least one embodiment of the present disclosure, the inert organic liquid phase may be chosen from polydecenes of formula C₁₀ₙH_{[(20n)+2]} in which n ranges from 3 to 9, for example, from 3 to 7, esters of fatty alcohols, esters of fatty acids, and mixtures thereof.

According to one embodiment of the present disclosure, the inert organic liquid phase may be present in the composition in an amount ranging from 5% to 60% by weight, for example, from 10% to 50% by weight, or from 15% to 45% by weight relative to the total weight of the paste.

According to another embodiment of the present disclosure, the composition in accordance with the present disclosure may be anhydrous.

As used herein, a composition is “anhydrous” when it has a water content of less than 1% by weight, for example, less than 0.5% by weight relative to the total weight of the composition.

According to a further embodiment of the present disclosure, the composition may also comprise hydrogen peroxide.

The pH of the composition comprising hydrogen peroxide may generally range from 3 to 11, for example, from 7 to 11.

The composition in accordance with the present disclosure may also comprise at least one additive conventionally used in cosmetics. The composition may thus comprise mineral or organic thickeners, for example, associative or non-associative, anionic, cationic, nonionic, or amphoteric thickening polymers; fillers such as clays; binders such as vinylpyrrolidone; lubricants, for instance polyol stearates, alkali metal stearates, and alkaline-earth metal stearates; hydrophilic silicas; hydrophobic silicas; pigments; dyes other than those of the present disclosure; matting agents, for instance titanium oxides; anionic, nonionic, cationic, amphoteric, or zwitterionic surfactants; antioxidants; penetrants; sequestrants; buffers; dispersants; film-forming agents; preserving agents; opacifiers; vitamins; fragrances; anionic, cationic, nonionic, amphoteric, or zwitterionic polymers; ceramides; and conditioning agents, for instance volatile or non-volatile, modified or unmodified silicones.

When the composition in accordance with the disclosure comprises hydrogen peroxide, it may also comprise at least one agent for controlling the release of oxygen, such as magnesium carbonate or oxide.

The additives and agents for controlling the release of oxygen as defined above may be present in an amount for each ranging from 0.01% to 40% by weight, for example, from 0.1% to 30% by weight relative to the total weight of the composition.

A person skilled in the art will take care to select the at least one optional additional compound such that the advantageous properties intrinsically associated with the composition in accordance with the present disclosure are not, or are not substantially, adversely affected by the envisaged addition.

Disclosed herein is also a method for bleaching and simultaneous dyeing keratin fibers comprising applying to the keratin fibers a composition in accordance with the present disclosure. In at least one embodiment, the composition may further comprise hydrogen peroxide.

Also disclosed herein is a multi-compartment device comprising at least two compositions in separate compartments, the mixing of which leads to a composition containing hydrogen peroxide in accordance with the present disclosure as defined above.

In one embodiment of the present disclosure, the device may comprise a first compartment that contains a composition (A) comprising, in a suitable dyeing medium, at least one dye as defined above, a second compartment that contains an anhydrous composition (B) comprising at least one peroxygenated salt and at least one alkaline agent as defined above, and a third compartment that contains an aqueous hydrogen peroxide composition (E).

According to another embodiment of the present disclosure, the device may comprise a first compartment that contains an anhydrous composition (C) comprising at least one dye as defined above, at least one peroxygenated salt, and at least one alkaline agent as defined above, and a second compartment that contains an aqueous hydrogen peroxide composition (E).

In a further embodiment of the present disclosure, the device may comprise a first compartment that contains an anhydrous composition (B) comprising at least one peroxygenated salt and at least one alkaline agent as defined above, and a second compartment that contains a composition (D) comprising, in a suitable dyeing medium, at least one dye as defined above and hydrogen peroxide.

The suitable dyeing medium for compositions (A) and (D) generally may comprise water or a mixture of water and at least one organic solvent to dissolve the compounds that are not sufficiently water-soluble. Examples of suitable organic solvents include, but are not limited to, C₁-C₄ lower alkanols such as ethanol and isopropanol; glycerol; glycols and glycol ethers, for instance 2-butoxyethanol, propylene glycol, and propylene glycol monomethyl ether; and aromatic alcohols, for instance benzyl alcohol and phenoxyethanol; similar products; and mixtures thereof.

The at least one solvent may be present in the composition in an amount ranging from 1% to 40% by weight, for example, from 5% to 30% by weight, relative to the total weight of the dye composition.

The composition (A), also known as the “booster”, may be formulated at acidic, neutral, or alkaline pH, the pH possibly ranging from 3 to 12, for example, from 4 to 11.

The composition (D) may have a pH of less than 7, the acidic pH ensuring the stability of the hydrogen peroxide in this composition.

The compositions (A) and (D) may be in various forms, chosen, for example, from liquids, creams, gels, and any other form that is suitable for dyeing keratin fibers.

The anhydrous compositions (B) and (C) may be in the form of a powder or a paste. In this case, they may also comprise an inert organic liquid phase as defined above.

The aqueous hydrogen peroxide composition (E) may have a pH of less than 7, the acidic pH ensuring the stability of the hydrogen peroxide in this composition.

The compositions (A), (B), (C), (D), and (E) may also contain various additives conventionally used in cosmetics, such as those described above.

The compositions (E) and (D) may also comprise agents for controlling the release of oxygen, as defined above.

The device in accordance with the present disclosure may be equipped with a means for applying the desired mixture to the hair, such as the devices described in French Patent No. 2 586 913.

Using this device, it is possible to bleach and simultaneously dye keratin fibers by means of a method in accordance with the present disclosure as defined above.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLE

Acid Red 35 was dissolved in an aqueous-alcoholic solution (80/20) to a concentration of 3.5 g %. Booster A was thus obtained.

This solution was added just before use to a mixture comprising Platine Précision B bleaching powder comprising 51.5% of a mixture of sodium, potassium, and magnesium persulfates in the presence of 4.2% of a mixture of sodium metasilicate and ammonium chloride and an oxidizing agent E comprising a 40-volumes aqueous hydrogen peroxide composition. The proportions of the bleaching powder B/oxidizing agent E/booster A mixture were, respectively, 1/2/0.5.

A portion of this mixture was applied immediately to a 1 g lock of natural hair containing 90% white hairs, and also to a lock of 2.7 g of natural chestnut-brown hair.

The remainder of this mixture was applied 20 minutes later to a lock of 1 g of natural hair containing 90% white hairs and also to a lock of 2.7 g of natural chestnut-brown hair.

In all cases, the treatment conditions were identical. Ten grams of the composition was used for each 1 g of hair.

After a leave-on time of 30 minutes, the locks were rinsed and then shampooed, rinsed again, and dried.

The results are given in the table below.

TINTS OBTAINED AFTER APPLYING THE COMPOSITIONS
OF THE PRESENT DISCLOSURE

| | Natural hair containing | Natural chestnut- |
|---|---|---|
| | 90% white hairs | brown hair |
| Immediate application | Pale pink | Chromatic nacreous |
| of the mixture | | coppery |
| Delayed application | Pale pink | Chromatic nacreous |
| of the mixture | | coppery |

The natural hair containing 90% white hairs was used here to exacerbate any possible change in tint.

It was found that the same tint was obtained when the mixture was applied immediately and when its application was delayed.

These results show that the compositions in accordance with the present disclosure are stable over time.

What is claimed is:

1. A composition for bleaching and simultaneously dyeing keratin fibers, comprising:
at least one dye chosen from 7-(6′-methylphenylazo)-1-acetamido-3,6-disulfo-8-hydroxynaphthalene and the addition salts thereof;
at least one peroxygenated salt; and
at least one alkaline agent.

2. The composition of claim 1, wherein the at least one dye is chosen from the sodium salts of 7-(6′-methylphenylazo)-1-acetamido-3,6-disulfo-8-hydroxynaphthalene and mixtures thereof.

3. The composition of claim 2, in which the at least one dye is chosen from Acid Red 35, Acid Red 55, and mixtures thereof.

4. The composition of claim 1, wherein the at least one dye is present in the composition in an amount ranging from 0.0001% to 10% by weight relative to the total weight of the composition.

5. The composition of claim 1, wherein the at least one peroxygenated salt is chosen from alkali metal persulfates, perborates, percarbonates, or peroxides, alkaline-earth metal persulfates, perborates, percarbonates, or peroxides, and mixtures thereof.

6. The composition of claim 5, wherein the at least one peroxygenated salt is chosen from persulfates, and mixtures thereof.

7. The composition of claim 6, wherein the at least one peroxygenated salt is chosen from sodium persulfate, potassium persulfate, ammonium persulfate, and mixtures thereof.

8. The composition of claim 1, wherein the at least one peroxygenated salt is present in the composition in an amount ranging from 10% to 70% by weight relative to the total weight of the composition.

9. The composition of claim 1, wherein the at least one alkaline agent is chosen from urea, ammonium chloride, ammonium sulfate, ammonium phosphate, ammonium nitrate, alkali metal silicates, phosphates, or carbonates, alkaline-earth metal silicates, phosphates, or carbonates, and mixtures thereof.

10. The composition of claim 1, wherein the at least one alkaline agent is present in the composition in an amount ranging from 0.01% to 40% by weight relative to the total weight of the composition.

11. The composition of claim 1, further comprising at least one inert organic liquid phase.

12. The composition of claim 11, wherein the at least one inert organic liquid phase is chosen from polydecenes of formula C₁₀ₙH_{[(20n)+2]}, in which n ranges from 3 to 9, esters of fatty alcohols, esters of fatty acids, C₁₂-C₂₄ fatty acid esters of sugars, C₁₂-C₂₄ fatty acid diesters of sugars, cyclic ethers, cyclic esters, silicone oils, mineral oils, plant oils, and mixtures thereof.

13. The composition of claim 12, wherein the at least one inert organic liquid phase is chosen from polydecenes of formula C₁₀ₙH_{[(20n)+2]}, in which n ranges from 3 to 9, esters of fatty alcohols, esters of fatty acids, and mixtures thereof.

14. The composition of claim 11, wherein the at least one inert organic liquid phase is present in the composition in an amount ranging from 5% to 60% by weight relative to the total weight of the composition.

15. The composition of claim 1, wherein the composition is anhydrous.

16. The composition of claim 1, further comprising hydrogen peroxide.

17. A method for bleaching and simultaneously dyeing keratin fibers, comprising applying a composition to the keratin fibers, wherein the composition comprises:
at least one dye chosen from 7-(6′-methylphenylazo)-1-acetamido-3,6-disulfo-8-hydroxynaphthalene and the addition salts thereof;
at least one peroxygenated salt; and
at least one alkaline agent.

18. A multi-compartment device comprising at least two compositions in separate compartments, the mixing of which leads to a composition comprising:
at least one dye chosen from 7-(6′-methylphenylazo)-1-acetamido-3,6-disulfo-8-hydroxynaphthalene and the addition salts thereof;
at least one peroxygenated salt;
at least one alkaline agent; and
hydrogen peroxide.

19. The device of claim 18, comprising a first compartment, a second compartment, and a third compartment, wherein
the first compartment contains a composition (A) comprising, in a suitable dyeing medium, at least one dye chosen from 7-(6′-methylphenylazo)-1-acetamido-3,6-disulfo-8-hydroxynaphthalene and the addition salts thereof;
the second compartment contains an anhydrous composition (B) comprising at least one peroxygenated salt and at least one alkaline agent; and
the third compartment contains an aqueous hydrogen peroxide composition (E).

20. The device of claim 18, comprising a first compartment and a second compartment, wherein
the first compartment contains an anhydrous composition (C) comprising at least one dye chosen from 7-(6′-methylphenylazo)-1-acetamido-3,6-disulfo-8-hydroxynaphthalene and the addition salts thereof, at least one peroxygenated salt, and at least one alkaline agent; and
the second compartment contains an aqueous hydrogen peroxide composition (E).

21. The device of claim 18, comprising a first compartment and a second compartment, wherein
the first compartment contains an anhydrous composition (B) comprising at least one peroxygenated salt and at least one alkaline agent; and
the second compartment contains a composition (D) comprising, in a suitable dyeing medium, hydrogen peroxide and at least one dye chosen from 7-(6′-methylphenylazo)-1-acetamido-3,6-disulfo-8-hydroxynaphthalene and the addition salts thereof.
